(19) United States
(12) Patent Application Publication
(10) Pub. No.: US 2010/0168605 A1
(43) Pub. Date: Jul. 1, 2010

(54) METHOD AND APPARATUS FOR MEDICAL MEASUREMENT AND COMMUNICATION

(75) Inventor: Ronald M. Aarts, Geldrop (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/063,258

(22) PCT Filed: Jul. 19, 2006
(86) PCT No.: PCT/IB06/52477

§ 371 Date: Feb. 8, 2008

Related U.S. Application Data
(60) Provisional application No. 60/706,397, filed on Aug. 8, 2005.

(51) Int. Cl. A61B 5/01; A61B 5/00; H04B 7/00

(52) U.S. Cl. 600/549; 600/300; 455/41.2

(57) ABSTRACT

A system for obtaining and communication patient physiological information includes one or more of patient measurement devices (112), patient record devices (100), and portable interface devices. The devices communicate via a wireless communications interface (110, 152, 162) such as a near field communication interface. The patient measurement devices (100) obtain physiological information on each of a plurality of patients. This information is stored on a patient record device (100) associated with each patient. The portable interface devices (114) are used to access the information.

The present invention relates to the field of measurement and communication of physiological parameters in medicine. The invention also finds application in connection with the measurement and communication of other information.

In a health care setting such as in a hospital or clinic, health care providers such as nursing assistants, nurses, physicians, are often required to obtain physiological information on a number of patients. The health care providers typically obtain periodic measurements of physiological parameters such as pulse rate, blood pressure, temperature, and the like for each patient. Generally, this information is obtained using physiological measurement devices suitable for the parameter of interest, such as pulse rate monitors, sphygmometers, thermometers, and the like.

The health care providers typically write the parameter values, together with other relevant information such as the time, other observations or comments, and the health care provider's name in a physical patient file. The file is often located near the patient, such as on a clipboard at the end of the patient's bed, and may be carried with the patient as he or she moves to different locations in a facility. Those wishing to review the information then consult with the file.

Unfortunately, however, the manual entry process can be error prone and time consuming. Moreover, the information is not readily accessible to other authorized personnel, who must generally have physical access to the patient file or otherwise obtain a copy should it become necessary to review the information. Moreover, it is generally necessary to maintain physical files for many patients, with the corresponding logistical difficulties associated with storing and providing access to the relevant information. The process for converting the information to digital form for storage on a computer system such as hospital information system/radiology information system (HIS/RIS system) is likewise error prone and time consuming.

Aspects of the present invention address these matters, and others.

According to a first aspect of the present invention, an apparatus includes a short range wireless communication interface and patient proximate means for determining the value of a physiological parameter of each of a plurality of patients in succession. The means for determining is physically coupled with the wireless communications interface for operation therewith, the wireless communication interface communicates output data to a receiving device, and the output data includes the physiological parameter value of a patient and a source identifier which identifies a source of the data.

According to a more limited aspect of the invention, the source identifier identifies a user associated with the apparatus.

According to another more limited aspect of the invention, the source identifier identifies a physiological parameter determined by the means for determining.

According to another more limited aspect of the invention, the source identifier identifies the apparatus.

According to another more limited aspect of the invention, the apparatus includes means for indicating to an operator that the wireless communication interface is in wireless communication with a second device. Operator confirmation is required for communicating the output data.

According to another more limited aspect of the invention, the apparatus includes a clock and the output data includes time information.

According to another more limited aspect of the invention, the wireless communication interface is a very short range wireless communication interface.

According to another more limited aspect of the invention, the wireless communication interface is a near field communication interface.

According to another more limited aspect of the invention, the means for determining includes a thermometer and the physiological parameter is temperature.

According to a second aspect of the present invention, a method includes the steps of using a first apparatus including a short range wireless communication interface to determine a value indicative of a physiological parameter of a patient, using the short range wireless communication interface to communicate the value to a second apparatus, and repeating the steps of using the first apparatus and using the short range wireless communication interface for each of a plurality of patients. The second apparatus is associated with the patient.

According to a more limited aspect of the invention, the wireless communication interface is an ultra short range wireless communication interface.

According to still more limited aspect of the invention, the ultra short range wireless communication interface communicates using magnetic field induction.

According to yet more limited aspect of the invention, the step of using the short range wireless communication interface includes communicating a parameter identifier to the second apparatus.

According to yet more limited aspect of the invention, the step of using the short range wireless communication interface includes communicating a user identifier to the second apparatus.

According to another limited aspect of the invention, the method includes indicating to a human that the wireless communication interface has established communication with the second apparatus and requiring human confirmation of the communication.

According to another aspect of the present invention, a medical apparatus includes a medical measurement device which generates an output value indicative of a physiological parameter of a patient and a short range wireless communication interface. The medical measurement device is adapted for use with succeeding ones of a plurality of patients. The communication interface communicates output information to a receiving device, and the output information includes the output value and a parameter identifier which identifies the physiological parameter.

According to a more limited aspect of the invention, the wireless communication interface is a very short range wireless communication interface.

According to another more limited aspect of the invention, the wireless communication interface and the medical measurement device are contained in a common housing.

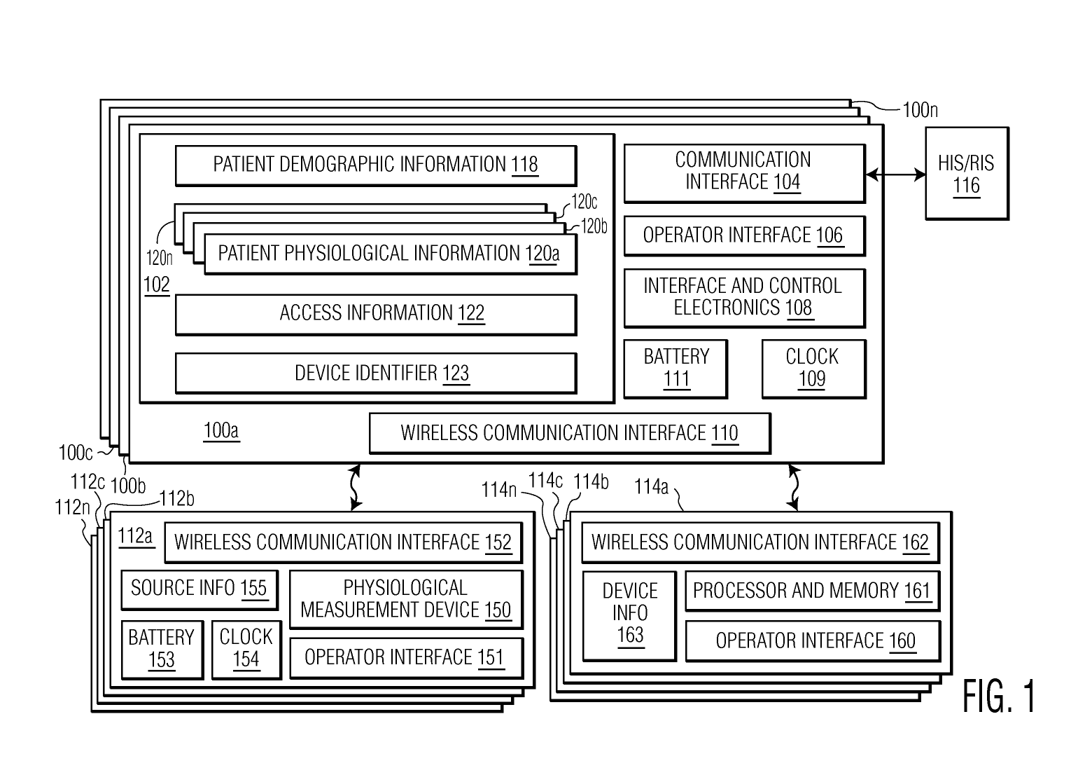

FIG. 1 depicts a physiological parameter measurement and communication system.

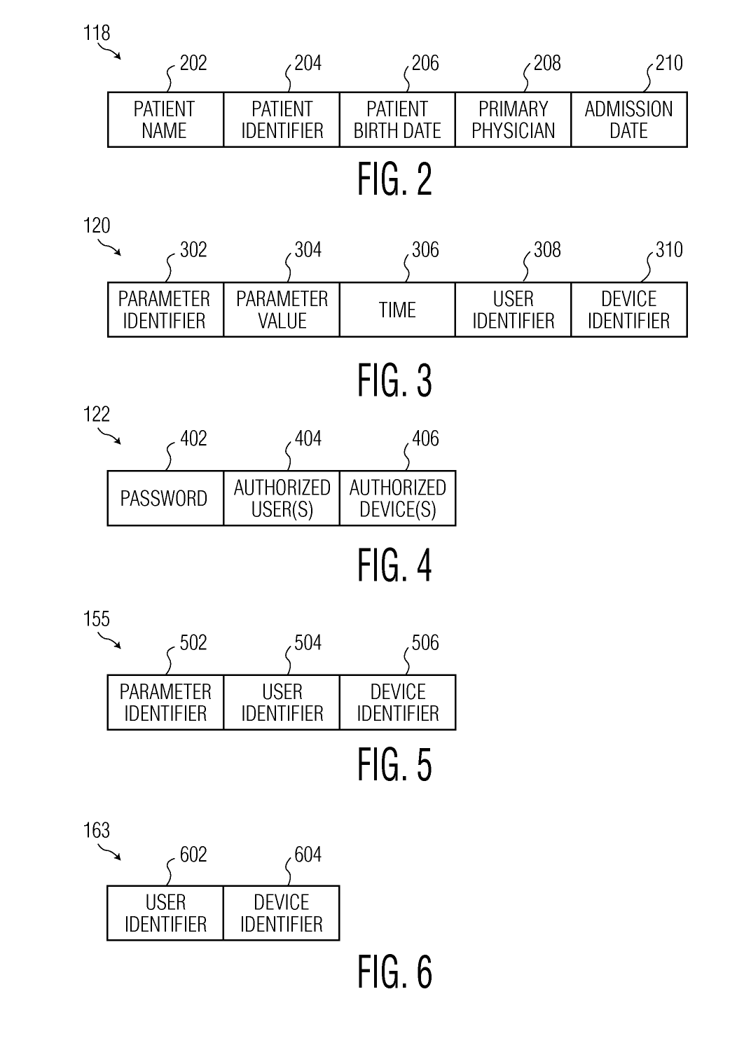

FIG. 2 depicts patient demographic information stored in patient record device.

FIG. 3 depicts patient physiological information stored in a patient record device.

FIG. 4 depicts access information stored in patient record device.

FIG. 5 depicts source information provided by a patient measurement device.

FIG. 6 depicts device information provided by a portable interface device.

With reference to FIG. 1, a system includes a plurality of patient record devices 100a, b, c . . . n, patient measurement devices 112a, b, c . . . n, and portable interface devices 114a, b, c . . . n.

Each patient record device 100 is preferably associated with a particular patient. The patient record device 100 is configured for removable attachment to the patient's anatomy using suitable straps, bands, necklaces or the like. The patient record device 100 may also be configured as a free standing device, in which case the device may be placed near the patient on a table, nightstand or the like, or it may be carried with the patient. The device may also be configured for removable attachment to the patient's bed or other structure near the patient, by way of suitable clamps or hooks, hook and loop fasteners, magnets, and the like. The device 100 may also be configured for more permanent mounting, for example to a wall or the patient's bed. The device also includes a location on which patient demographic information can be written or for receiving a sticker on which patient demographic information is printed.

The patient record device 100 includes computer readable memory 102, a communication interface 104, an operator interface 106, interface and control electronics 108, a wireless communications interface 110, a power supply 111, and a clock 109.

The wireless communication interface 110 is preferably an ultra short range interface having a maximum range less than about 0.3 meters (1 foot). The wireless communications interface may also be a very short range interface having a maximum range on the order of a typical bedside, or approximately 2 meters (6.6 feet), or a short range communication interface having maximum range on the order of the distances encountered in a typical hospital room, or approximately 4 meters (13 feet).

In one embodiment, the wireless communications interface 110 is a near field communications (NFC) interface. As is known in the art, NFC interfaces operate through magnetic field induction and have a range on the order 10 centimeters (3.9 inches). However, other suitable interfaces may also be used, including those based on electromagnetic and optical technologies. Commercially available examples of the former include Bluetooth, wireless fidelity (wi-fi), and HIPERLAN technology; examples of the latter include infrared techniques such as those based on the Infrared Data Association (IrDA) standard. Of course, other standard or proprietary interfaces may be implemented.

The memory 102 is preferably a non-volatile, alterable memory such as electrically erasable programmable read only memory, flash memory, battery-backed CMOS static RAM. Of course, other technologies, such as nanotube memory, magnetic or ferromagnetic memory could also be implemented. The memory may also take various forms, such as one or more removable memory cards or sticks, or it may be a permanent or semi-permanent part of the device. The memory 102 stores information including patient demographic information 118, patient physiological information elements 120a, b, c . . . n, access information 122, and a device identifier or ID number 123.

With reference to FIGS. 1 and 2, the patient demographic information 118 includes the patient's name 202, a patient identifier such as an identification number 204, the patient's birth date or age 206, physician information such as the name of the patient's primary physician 208, and admission information such as the patient's admission date or number 210. Of course, the memory 102 may also store additional or different patient demographic information 118. For security purposes, it may be undesirable to store some or all of the patient demographic information 118 in the memory 102. In such a case, more complete demographic information can be obtained by using the patient identification number 204 in connection with a separate database. Additionally, it may be desirable to encrypt some or all of the information stored in the memory 102. With reference to FIGS. 1 and 3, the memory 102 stores a plurality of patient physiological information elements 120a, b, c . . . n. Each element 120 includes one or more of a physiological parameter identifier 302, a physiological parameter value 304, a time 306, an operator identifier 308, and a device identifier 310. It should be noted that the various information contained in the elements 120 (or for that matter in the memory 102 in general) need not be stored in contiguous memory locations but may instead be stored using suitable data structures.

The parameter identifier 302 identifies the parameter which is the subject of the particular element 120. Example parameters include physiological parameters such as temperature, pulse, respiration rate, blood pressure, blood oxygen saturation, weight, and bone density. Other parameters may include notes or observations such as those entered by a physician or nurse. The parameter value 304 is the value of the particular parameter 302. Where the parameter identifier 302 specifies a temperature, for example, the parameter value 304 is the value of the patient's temperature. The time 306 includes time information such as a time and/or date associated with the element 120. The user identifier 308 identifies a health care provider associated with the particular element 120. The device identifier 120 identifies a device associated with the element 120.

With reference to FIGS. 1 and 4, the access information 122 is used to limit access to the device and may include one or more of a password 402, a user identifier 404 which identifies one or more users who are authorized to access the device 100, and a device identifier 406 which identifies one or more devices such as patient measurement devices 112 or portable interface devices 114 which are authorized to access the device 100.

The patient record device identifier 123 is an identification code or serial number which identifies the patient record device 100.

Returning now to FIG. 1, the operator interface 106 preferably includes an audible output such as a buzzer or beeper and a visual output such as one or more light emitting diodes (LEDs), liquid crystal displays (LCDs), or numeric or alphanumeric displays. The operator interface 106 also includes an input device such as buttons or keys.

The computer interface 104 provides a connection with one or more computing devices over a suitable connection such as a local area network (LAN) or wide area network (WAN). The interface 104 is preferably selected for compatibility with a particular hospital or health care facility network. Typical interface technologies include

Ethernet and wireless technologies such as Bluetooth or wi-fi. Of course, other standard or proprietary interfaces may be implemented.

Interface and control electronics 108 provide the necessary connections and control among the various elements of the patient record device 100.

A power supply 111 such as a battery provides power to the device 100. The power supply may also be connected to the electrical power mains. The clock 109 provides time information, such as the current time and/or date.

With continuing reference to FIG. 1, each patient measurement device 112 includes a physiological measurement device 150, an operator interface 151, a wireless communication interface 152, a power supply 153, and a clock 154. Also associated with each unit 112 is source information 155.

The physiological measurement devices 112 are not necessarily associated with a particular patient, and are preferably suitable for use with a number of patients in succession. Depending on the specifics of the unit 112 and the situation, a particular unit 112 may be associated with a particular health care provider or it may be generally accessible to health care providers working on a particular floor or at a particular institution.

The physiological measurement device 150 provides an output which is indicative of a physiological parameter of a patient. Various physiological measurement devices 150 are well known and include devices such as thermometers, sphygmometers, heart rate monitors, pulse oximeters, respiration monitors, apnea monitors, scales, bone density monitors, and the like. Of course, the above devices 150 are exemplary only and other suitable devices are known and may be used in connection with the patient measurement device 100.

Preferably, the physiological measurement device 150 is adapted for use with each of a plurality of patients in succession. By way of example, thermometers are typically portable and are readily brought into proximity with a particular patient to be examined. In addition, thermometers are typically provided with disposable sleeves, or can otherwise be suitably cleaned between patients, to avoid the spread of pathogens. As another example, scales often remain stationary while successive patients are brought to the scale for measurement. It should also be noted that some physiological measurement devices 150 may include a sensor which typically remains affixed to a particular patient while the remainder of the device 150 is used with a plurality of patients. In that case, it may be desirable to use a suitable connector which allows the sensor to be selectively attached and detached.

The operator interface 150 preferably includes an audible output such as a buzzer or beeper and a visual output such as one or more light emitting diodes (LEDs), liquid crystal display (LCDs), or numeric or alphanumeric displays. The operator interface 150 also includes an input device such as buttons or keys.

The wireless communication interface 152 is selected for compatibility with the wireless communication interface 110 of the patient record devices 100. As noted above, one suitable interface is an NFC interface. Other suitable ultra short range, very short range, or short range communications interfaces may also be implemented as described above.

In the case of a readily portable patient measurement device 112, the physiological measurement device 150, operator interface 151, and wireless communication interface 152 are preferably mounted together as a single unit or in a single housing. Alternately the wireless communications interface 152 may be mounted separately from the measurement device 150 or the operator interface 151 and physically coupled with them using a flexible cable or other suitable wiring. Such a configuration is particularly advantageous in situations where the measurement device 112 is stationary or otherwise less portable, as it allows the wireless communications interface 152 to be placed in a relatively convenient location.

A power supply 153 such as a battery provides power to the patient measurement unit 112. Power may also be provided by a power supply connected to the electrical power mains. The clock 154 provides time information, such as the current time and/or date.

With reference to FIGS. 1 and 5, source information 155 is associated with each patient measurement device 112. The information includes one or more of a physiological parameter identifier 502 which identifies the parameter or parameters measured by the measurement device 150, a user identifier 504 which identifies a health care provider associated with the particular unit measurement device 112, and a device identifier 506 such as a designator or serial number which identifies the device 112. This information is preferably stored in a suitable memory.

With continuing reference to FIG. 1, the portable interface devices 114a, b, c . . . n are preferably personal digital assistants (PDAs), tablet computers, laptop or notebook computers, or other suitable portable computing devices. Each portable interface device 114 is typically associated with a particular health care provider and includes an operator interface 160 such as a display, keyboard, audio input and output, and/or stylus for communicating with the user as well as a memory and processor 161 for storing and executing one or more computer programs. Each portable interface also includes a wireless communication interface 162. Also associated with each device 114 is device information 163.

The wireless communication interface 162 is again selected for compatibility with the wireless communication interface 110 associated with the patient record devices 100. Alternately, the communication interface 162 may be selected for compatibility with the computer interface 104.

With reference to FIGS. 1 and 6, the device information 163 includes information which serves to identify the device or its user. A user identifier 602 identifies a user associated with the particular device 114. A device identifier 604 such as a designator or serial number identifies the device 114.

The memory 161 contains instructions which, when executed by the processor, allow the user to store, view or otherwise manipulate information received by the wireless communications interface 162. In one embodiment, the user may use the interface device 114 to receive information stored in a desired patient record device 100. Relevant patient demographic information 118 is displayed on the operator interface 160. This information allows the user to confirm that he or she has accessed information on the desired patient and to obtain basic demographic information. The various patient physiological information elements 120 including relevant parameter identifiers 302, parameter values 304, time information 306, user identifiers 308, and device identifiers 310 may also be displayed. Preferably, the program allows the user to filter or sort the elements 120, for example by the time or date 306, the parameter identifier 302, the user identifier 308, or the like. Similarly, the user may also elect to view only certain information, such as information which includes a particular parameter identifier 302, where the parameter value 304 is outside of specified range, or the like. The latter is particularly useful where the user desires to readily locate information which requires scrutiny, such as an elevated temperature or a blood pressure reading which lies outside of a normal range.

The device 114 may also allow the operator to enter relevant patient demographic information 118 for transfer to a patient record device 102 using the wireless communication interface 110. This is particularly useful for associating a device 110 with a particular patient.

Moreover, the device 114 may also allow the user to enter notes, comments, or other information through the operator interface 160 and transfer the information to a particular patient record device using the wireless communication interface 110 in a manner analogous to the transfer from a patient measurement unit 112 to a patient record device 100.

In operation, a patient record device 100 is initialized and associated with a particular patient. Patient demographic information 118 is received by the device through the wireless communication interface 110 and stored in memory 102. Alternately, the information may be received via the communications interface 104 or entered via the operator interface 106. Access information 122 and the current time information is similarly provided and stored. The user may also wish to affix some or all of the patient demographic information to the device 100, for example by placing a sticker or writing the information on the device.

The device 112 is attached to a suitable portion of the patient's anatomy, for example by strapping it to the patient's arm or leg, or by placing it around the patient's neck using a necklace. Alternately, the device is placed in a suitable location near the patient such as at the end of the patient's bed, on stand or table located on the patient's bed, clipped to a clipboard, or the like.

Of course, additional patient record devices 100 may be initialized and associated with other patients.

In a clinical setting, a health care provider is usually required to obtain and record physiological parameter measurements for one or more patients. Depending on the parameter or parameters of interest, the health care provider obtains the appropriate measurement device or devices 112. If not already done, the health care provider enters the correct user identifier 504 through the operator interface 151 or the wireless communications interface 152. The health care provider then brings the device 112 into proximity to the desired patient. Alternately, and particularly in the case of less portable units 112, the patient is brought to the unit 112. The health care provider then uses the measurement device 150 to obtain the value of the desired physiological parameter. Depending on the device 150, the operator interface 151 may provide a visual, audible, or other signal indicating that the measurement is complete, and may also display the relevant parameter value or values. Information from the clock 154 is used to establish the time that the measurement was obtained. The relevant information is also made available to the wireless communication interface 110.

The health care provider then brings the wireless communications interfaces 110, 152 of the patient record device 100 and the measurement unit 112 within communications range. Where the communications interfaces 152, 100 are NFC interfaces, the respective interfaces must be located from touching or near touching to a maximum distance of about 10 centimeters (3.9 inches). The NFC interfaces preferably operate in the so-called touch and confirm mode. Accordingly, the operator interface 106 of the patient record device 100 indicates that communications have been established with a physiological measurement unit 112 by beeping, flashing a visible indicator, or the like. The health care provider confirms the transfer through the operator interface 106 on the patient record device 100, for example by pressing a button. Alternately, the indication and confirmation may be accomplished through the operator interface 151 of the physiological measurement unit 112, or through a combination of both operator interfaces. Still alternately, the interfaces 110, 152 may operate in the so-called touch and go mode, wherein one or both of the indication or confirmation is not required. The patient record device 100 also compares the access information 122 against the source information 155. If the measurement unit 112 or its user is not authorized to access the patient record device 100, then the information transfer is refused, and the attempted access is logged in the memory 102. The access attempt may also be the subject of an alarm, for example through the operator interface 106 or through the HIS/RIS system 116 or other computer.

In any case, the source information 155, parameter value 304, and time 306 are transmitted by the wireless communications interface 112 and received by the wireless communications interface 110 associated with the patient record device 100. The information is stored in the memory 102.

Depending on the situation, the health care provider may decide to use the patient measurement device 112 to obtain information relating to successive patients, either immediately, over the course of a shift or other period, or as otherwise needed. In any case, the information is obtained and transferred to the desired patient record device 100 for each patient.

Where a patient record device 100 is connected to a HIS/RIS system 116 or other computer through the computer interface, information contained in the memory 102 is also transferred to the HIS/RIS system 116 or other computer.

A health care provider, such as a physician or nurse on rounds, may wish to use a portable interface device 114 to view or otherwise manipulate information contained in a particular patient record device 100. As generally describe above, communications are established between the portable interface device 114 and the desired patient record device 100, and the information is transferred via the respective communication interfaces. If the device information 163 does not match the access information 122, the information transfer is refused.

Of course, various alternatives are also possible. For example, some or all of the information contained in the memory 120 may be omitted. Similarly, the clock 109, 154 may be omitted from one or both of the patient measurement devices 112 or the patient record devices 100. Where the clock 154 is omitted from the patient measurement devices 112, the time information 306 stored in the memory 120 is preferably the time at which a particular information element 120 was received. Moreover, where the system includes only like measurement units 150 which provide values for only a single parameter (or a known plurality of parameters), then the parameter identifiers 302 may be omitted. Of course, still other choices of information may be implemented.

The invention has been described with reference to the preferred embodiments. Of course, modifications and alterations will occur to others upon reading and understanding the preceding description. It is intended that the invention be construed as including all such modifications and alterations insofar as they come within the scope of the appended claims or the equivalents thereof.

1. An apparatus comprising:
a short range wireless communication interface (152); and
patient proximate means (150) for determining the value of a physiological parameter of each of a plurality of patients in succession;
wherein the means (150) for determining is physically coupled with the wireless communications interface (152) for operation therewith, wherein the wireless communication interface (152) communicates output data to a receiving device (100), and wherein the output data includes the physiological parameter value of a patient and a source identifier which identifies a source of the data.

2. The apparatus of claim 1 wherein the source identifier identifies a user associated with the apparatus.

3. The apparatus of claim 1 wherein the source identifier identifies a physiological parameter determined by the means for determining.

4. The apparatus of claim 1 wherein the source identifier identifies the apparatus.

5. The apparatus of claim 1 wherein the apparatus includes means (151) for indicating to an operator that the wireless communication interface (152) is in wireless communication with a second device and wherein operator confirmation is required for communicating the output data.

6. The apparatus of claim 1 wherein the apparatus includes a clock (154) and the output data includes time information.

7. The apparatus of claim 1 wherein the wireless communication interface (152) is a very short range wireless communication interface.

8. The apparatus of claim 1 wherein the wireless communication interface (152) is a near field communication interface.

9. The apparatus of claim 1 wherein the means for determining (150) includes a thermometer and the physiological parameter is temperature.

10. A method comprising:
using a first apparatus (112) including a short range wireless communication interface (152) to determine a value indicative of a physiological parameter of a patient;
using the short range wireless communication interface (152) to communicate the value to a second apparatus (100), wherein the second apparatus is associated with the patient;
repeating the steps of using the first apparatus (112) and using the short range wireless communication interface (152) for each of a plurality of patients.

11. The method of claim 10 wherein the wireless communication interface (152) is an ultra short range wireless communication interface.

12. The method of claim 11 wherein the ultra short range wireless communication interface (152) communicates using magnetic field induction.

13. The method of claim 12 wherein the step of using the short range wireless communication interface (152) includes communicating a parameter identifier to the second apparatus.

14. The method of claim 13 wherein the step of using the short range wireless communication interface (152) includes communicating a user identifier to the second apparatus.

15. The method of claim 10 further including indicating to a human that the wireless communication interface (152) has established communication with the second apparatus (100) and requiring human confirmation of the communication.

16. A medical apparatus comprising:
a medical measurement device (150) which generates an output value indicative of a physiological parameter of a patient, wherein the device is adapted for use with succeeding ones of a plurality of patients;
a short range wireless communication interface (152), wherein the wireless communication interface communicates output information to a receiving device (100), and wherein the output information includes the output value and a parameter identifier which identifies the physiological parameter.

17. The apparatus of claim 16 wherein the wireless communications interface (152) includes a near field communication interface.

18. The apparatus of claim 17 wherein the near field communication interface (152) operates in the touch and confirm mode.

19. The apparatus of claim 16 wherein the wireless communication interface (152) is a very short range wireless communication interface.

20. The apparatus of claim 16 wherein the wireless communication interface (152) is an ultra short range wireless communication interface.

21. The apparatus of claim 16 wherein the output information includes information which identifies the apparatus.

22. The apparatus of claim 16 wherein the output information includes information which identifies a health care provider associated with the apparatus.

23. The apparatus of claim 16 wherein the wireless communication interface (152) and the medical measurement device (150) are contained in a common housing.
